(19) United States
(12) Patent Application Publication
(10) Pub. No.: US 2008/0253967 A1
(43) Pub. Date: Oct. 16, 2008

(54) Halo-Stilbene Derivatives And Their Use For Binding And Imaging Of Amyloid Plaques

(76) Inventors: Hank F. Kung, Wynnewood, PA (US); Mei Ping Kung, Wynnewood, PA (US); Rajesh Manchanda, Morton, PA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 12/101,275

(22) Filed: Apr. 11, 2008

Related U.S. Application Data
(60) Provisional application No. 60/907,702, filed on Apr. 13, 2007.

(51) Int. Cl. A61K 31/136; C07C 217/80; A61K 49/00

(52) U.S. Cl. 424/9.1; 564/346; 514/652

(57) ABSTRACT

This invention relates to a method of imaging amyloid deposits and to styrylpyridine compounds, and methods of making radiolabeled styrylpyridine compounds useful in imaging amyloid deposits. This invention also relates to compounds, and methods of making compounds for inhibiting the aggregation of amyloid proteins to form amyloid deposits, and a method of delivering a therapeutic agent to amyloid deposits.

### CROSS REFERENCE TO RELATED APPLICATIONS

This application claims the benefit of U.S. Provisional Application No. 60/907,702, filed Apr. 13, 2007, the entirety of which is incorporated herein by reference.

### STATEMENT REGARDING FEDERALLY-SPONSORED RESEARCH

Part of the work performed during development of this invention utilized U.S. Government funds. The U.S. Government has certain rights in this invention under grant number AG-022559 awarded by the National Institutes of Health.

### FIELD OF THE INVENTION

This invention relates to novel styrylpyridine compounds, the uses thereof in diagnostic imaging and inhibiting amyloid-β aggregation, and methods of making these compounds.

### BACKGROUND OF THE INVENTION

Alzheimer's disease (AD) is a progressive neurodegenerative disorder characterized by cognitive decline, irreversible memory loss, disorientation, and language impairment. Postmortem examination of AD brain sections reveals abundant senile plaques (SPs) composed of amyloid-β (Aβ) peptides and numerous neurofibrillary tangles (NFTs) formed by filaments of highly phosphorylated tau proteins (for recent reviews and additional citations see Ginsberg, S. D., et al., “Molecular Pathology of Alzheimer's Disease and Related Disorders,” in Cerebral Cortex: Neurodegenerative and Age-related Changes in Structure and Function of Cerebral Cortex, Kluwer Academic/Plenum, N.Y. (1999), pp. 603-654; Vogelsberg-Ragaglia, V., et al., “Cell Biology of Tau and Cytoskeletal Pathology in Alzheimer's Disease,” Alzheimer's Disease, Lippincot, Williams & Wilkins, Philadelphia, Pa. (1999), pp. 359-372).

Amyloidosis is a condition characterized by the accumulation of various insoluble, fibrillar proteins in the tissues of a patient. An amyloid deposit is formed by the aggregation of amyloid proteins, followed by the further combination of aggregates and/or amyloid proteins. Formation and accumulation of aggregates of \tilde{β} amyloid (A [symbol] peptides in the brain are critical factors in the development and progression of AD.

In addition to the role of amyloid deposits in Alzheimer's disease, the presence of amyloid deposits has been shown in diseases such as Mediterranean fever, Muckle-Wells syndrome, idiopathic myeloma, amyloid polyneuropathy, amyloid cardiomyopathy, systemic senile amyloidosis, amyloid polyneuropathy, hereditary cerebral hemorrhage with amyloidosis, Down's syndrome, Scrapie, Creutzfeldt-Jacob disease, Kuru, Gerstamnn-Straussler-Scheinker syndrome, medullary carcinoma of the thyroid, Isolated atrial amyloid, β₂-microglobulin amyloid in dialysis patients, inclusion body myositis, β₂-amyloid deposits in muscle wasting disease, and Islets of Langerhans diabetes Type II insulinoma.

The fibrillar aggregates of amyloid peptides, Aβ₁₋₄₀ and Aβ₁₋₄₂, are major metabolic peptides derived from amyloid precursor protein found in senile plaques and cerebrovascular amyloid deposits in AD patients (Xia, W., et al., J. Proc. Natl. Acad. Sci. U.S.A. 97:9299-9304 (2000)). Prevention and reversal of Aβ plaque formation are being targeted as a treatment for this disease (Selkoe, D., J. JAMA 283:1615-1617 (2000); Wolfe, M. S., et al., J. Med. Chem. 41:6-9 (1998); Skovronsky, D. M., and Lee, V. M., Trends Pharmacol. Sci. 21:161-163 (2000)).

Familial AD (FAD) is caused by multiple mutations in the A precursor protein (APP), presenilin 1 (PS1) and presenilin 2 (PS2) genes (Ginsberg, S. D., et al., “Molecular Pathology of Alzheimer's Disease and Related Disorders,” in Cerebral Cortex: Neurodegenerative and Age-related Changes in Structure and Function of Cerebral Cortex, Kluwer Academic/Plenum, N.Y. (1999), pp. 603-654; Vogelsberg-Ragaglia, V., et al., “Cell Biology of Tau and Cytoskeletal Pathology in Alzheimer's Disease,” Alzheimer's Disease, Lippincot, Williams & Wilkins, Philadelphia, Pa. (1999), pp. 359-372).

While the exact mechanisms underlying AD are not fully understood, all pathogenic FAD mutations studied thus far increase production of the more amyloidogenic 42-43 amino-acid long form of the Aβ peptide. Thus, at least in FAD, dysregulation of Aβ production appears to be sufficient to induce a cascade of events leading to neurodegeneration. Indeed, the amyloid cascade hypothesis suggests that formation of extracellular fibrillar Aβ aggregates in the brain may be a pivotal event in AD pathogenesis (Selkoe, D. J., “Biology of β-amyloid Precursor Protein and the Mechanism of Alzheimer's Disease,” Alzheimer's Disease, Lippincot Williams & Wilkins, Philadelphia, Pa. (1999), pp. 293-310; Selkoe, D. J., J. Am. Med. Assoc. 283:1615-1617 (2000); Naslund, J., et al., J. Am. Med. Assoc. 283:1571-1577 (2000); Golde, T. E., et al., Biochimica et Biophysica Acta 1502:172-187 (2000)).

Various approaches in trying to inhibit the production and reduce the accumulation of fibrillar Aβ in the brain are currently being evaluated as potential therapies for AD (Skovronsky, D. M. and Lee, V. M., Trends Pharmacol. Sci. 21:161-163 (2000); Vassar, R., et al., Science 286:735-741 (1999); Wolfe, M. S., et al., J. Med. Chem. 41:6-9 (1998); Moore, C. L., et al., J. Med. Chem. 43:3434-3442 (2000); Findeis, M. A., Biochimica et Biophysica Acta 1502:76-84 (2000); Kuner, P., Bohrmann, et al., J. Biol. Chem. 275:1673-1678 (2000)). It is therefore of interest to develop ligands that specifically bind fibrillar Aβ aggregates. Since extracellular SPs are accessible targets, these new ligands could be used as in vivo diagnostic tools and as probes to visualize the progressive deposition of Aβ in studies of AD amyloidogenesis in living patients.

To this end, several interesting approaches for developing fibrillar Aβ aggregate-specific ligands have been reported (Ashburn, T. T., et al., Chem. Biol. 3:351-358 (1996); Han, G., et al., J. Am. Chem. Soc. 118:4506-4507 (1996); Klunk, W. E., et al., Biol. Psychiatry 35:627 (1994); Klunk, W. E., et al., Neurobiol. Aging 16:541-548 (1995); Klunk, W. E., et al., Society for Neuroscience Abstract 23:1638 (1997); Mathis, C. A., et al., Proc. XIIth Intl. Symp. Radiopharm. Chem., Uppsala, Sweden:94-95 (1997); Lorenzo, A. and Yankner, B. A., Proc. Natl. Acad. Sci. U.S.A. 91:12243-12247 (1994); Zhen, W., et al., J. Med. Chem. 42:2805-2815 (1999)). The most attractive approach is based on highly conjugated chrysamine-G (CG) and Congo red (CR), and the latter has been used for fluorescent staining of SPs and NFTs in postmortem AD brain sections (Ashburn, T. T., et al., Chem. Biol. 3:351-358 (1996); Klunk, W. E., et al., J. Histochem. Cytochem. 37:1273-1281 (1989)). The inhibition constants (Kᵢ) for binding to fibrillar Aβ aggregates of CR, CG, and 3′-bromo- and 3′-iodo derivatives of CG are 2,800, 370, 300 and 250 nM, respectively (Mathis, C. A., et al., Proc. XIIth Intl. Symp. Radiopharm. Chem., Uppsala, Sweden:94-95 (1997)). These compounds have been shown to bind selectively to Aβ (1-40) peptide aggregates in vitro as well as to fibrillar Aβ deposits in AD brain sections (Mathis, C. A., et al., Proc. XIIth Intl. Symp. Radiopharm. Chem., Uppsala, Sweden:94-95 (1997)).

There are several potential benefits of imaging Aβ aggregates in the brain. The imaging technique will improve diagnosis by identifying potential patients with excess Aβ plaques in the brain; therefore, they may be likely to develop Alzheimer's disease. It will also be useful to monitor the progression of the disease. When anti-plaque drug treatments become available, imaging Aβ plaques in the brain may provide an essential tool for monitoring treatment. Thus, a simple, noninvasive method for detecting and quantitating amyloid deposits in a patient has been eagerly sought. Presently, detection of amyloid deposits involves histological analysis of biopsy or autopsy materials. Both methods have drawbacks. For example, an autopsy can only be used for a postmortem diagnosis.

The direct imaging of amyloid deposits in vivo is difficult, as the deposits have many of the same physical properties (e.g., density and water content) as normal tissues. Attempts to image amyloid deposits using magnetic resonance imaging (MRI) and computer-assisted tomography (CAT) have been disappointing and have detected amyloid deposits only under certain favorable conditions. In addition, efforts to label amyloid deposits with antibodies, serum amyloid P protein, or other probe molecules have provided some selectivity on the periphery of tissues, but have provided for poor imaging of tissue interiors.

Potential ligands for detecting Aβ aggregates in the living brain must cross the intact blood-brain barrier. Thus brain uptake can be improved by using ligands with relatively smaller molecular size (compared to Congo Red) and increased lipophilicity. Highly conjugated thioflavins (S and T) are commonly used as dyes for staining the Aβ aggregates in the AD brain (Elhaddaoui, A., et al., Biospectroscopy 1:351-356 (1995)).

A highly lipophilic tracer, [¹⁸F]FDDNP, for binding both tangles (mainly composed of hyperphosphorylated tau protein) and plaques (containing Aβ protein aggregates) has been reported. (Shoghi-Jadid K, et al., Am J Geriatr Psychiatry. 2002; 10:24-35). Using positron-emission tomography (PET), it was reported that this tracer specifically labeled deposits of plaques and tangles in nine AD patients and seven comparison subjects. (Nordberg A. Lancet Neurol. 2004; 3:519-27). Using a novel pharmacokinetic analysis procedure called the relative residence time of the brain region of interest versus the pons, differences between AD patients and comparison subjects were demonstrated. The relative residence time was significantly higher in AD patients. This is further complicated by an intriguing finding that FDDNP competes with some NSAIDs for binding to Aβ fibrils in vitro and to Aβ plaques ex vivo (Agdeppa E D, et al. 2001; Agdeppa E D, et al., Neuroscience. 2003; 117:723-30).

Imaging β-amyloid in the brain of AD patients by using a benzothiazole aniline derivative, [¹¹C]6-OH-BTA-1 (also referred to as [¹¹C]PIB), was recently reported. (Mathis C A, et al., Curr Pharm Des. 2004; 10:1469-92; Mathis C A, et al., Arch. Neurol. 2005, 62:196-200.). Contrary to that observed for [¹⁸F]FDDNP, [¹¹C]6-OH-BTA-1 binds specifically to fibrillar A [symbol] in vivo. Patients with diagnosed mild AD showed marked retention of [¹¹C]6-OH-BTA-1 in the cortex, known to contain large amounts of amyloid deposits in AD. In the AD patient group, [¹¹C]6-OH-BTA-1 retention was increased most prominently in the frontal cortex. Large increases also were observed in parietal, temporal, and occipital cortices and in the striatum. [¹¹C]6-OH-BTA-1 retention was equivalent in AD patients and comparison subjects in areas known to be relatively unaffected by amyloid deposition (such as subcortical white matter, pons, and cerebellum). Recently, another ¹¹C. labeled A [symbol] plaque-targeting probe, a stilbene derivative-[¹¹C]SB-13, has been studied. In vitro binding using the [³H]SB-13 suggests that the compound showed excellent binding affinity and binding can be clearly measured in the cortical gray matter, but not in the white matter of AD cases. (Kung M-P, et al., Brain Res. 2004; 1025:89-105. There was a very low specific binding in cortical tissue homogenates of control brains. The Kd values of [³H]SB-13 in AD cortical homogenates were 2.4±0.2 nM. High binding capacity and comparable values were observed (14-45 μmol/mg protein) (Id.). As expected, in AD patients [¹¹C]SB-13 displayed a high accumulation in the frontal cortex (presumably an area containing a high density of Aβ plaques) in mild to moderate AD patients, but not in age-matched control subjects. (Verhoeff N P, et al., Am J Geriatr Psychiatry. 2004; 12:584-95).

It would be useful to have a noninvasive technique for imaging and quantitating amyloid deposits in a patient. In addition, it would be useful to have compounds that inhibit the aggregation of amyloid proteins to form amyloid deposits and a method for determining a compound's ability to inhibit amyloid protein aggregation.

### SUMMARY OF THE INVENTION

The present invention is directed to compounds of Formula I. The present invention also provides diagnostic compositions comprising radiolabeled compounds of Formula I and a pharmaceutically acceptable carrier or diluent.

The invention further provides methods of imaging amyloid deposits, the methods comprising introducing into a patient a detectable quantity of a labeled compound of Formula I or a pharmaceutically acceptable salt, ester, amide or prodrug thereof.

The present invention also provides a method for inhibiting the aggregation of amyloid proteins, the method comprising administering to a mammal an amyloid inhibiting amount of a compound Formula I or a pharmaceutically acceptable salt, ester, amide, or prodrug thereof.

A further aspect of this invention is directed to methods and intermediates useful for synthesizing the amyloid inhibiting and imaging compounds of Formula I described herein.

### BRIEF DESCRIPTION OF THE DRAWINGS

FIG. 1 depicts the chemical structures of certain embodiments of the present invention.

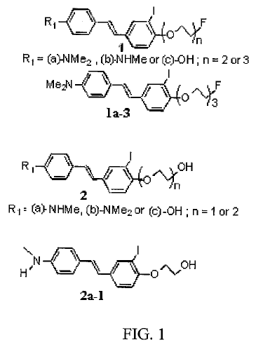

FIG. 2 depicts the chemical structure of a preferred embodiment of the present invention and certain binding data.

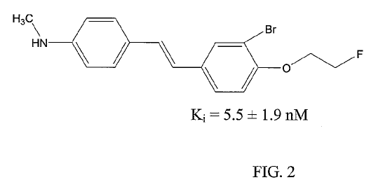

### DETAILED DESCRIPTION OF ILLUSTRATIVE EMBODIMENTS

A compound of Formula I,

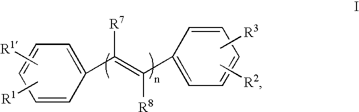

or a pharmaceutically acceptable salt thereof, wherein,

n is an integer from one to six;

R¹ and R^{1†} are each independently:
-(CH₂)ₚNR^{a}R^{b}, wherein R^{a} and R^{b} are independently hydrogen, (C₁₋₄)alkyl, hydroxy(C₁₋₄)alkyl or halo(C₁₋₄)alkyl, and p is an integer from 0 to 5; hydroxy, (C₁₋₄)alkoxy; hydroxy(C₁₋₄)alkyl; halogen; cyano; hydrogen; nitro; (C₁-C₄)alkyl; halo(C₁-C₄)alkyl; formyl; —NHCO(C₁₋₄ alkyl); or —OCO(C₁₋₄) alkyl;

in all embodiments, it is preferred that one of R¹ and R^{1†} is other than hydrogen;

R² is:

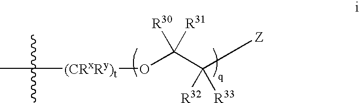

wherein q is an integer from 1 to 10; R^{x} and R^{y} are each hydrogen, hydroxy or (C₁₋₄)alkyl; t is 0, 1, 2 or 3; Z is halogen, halogen substituted benzoyloxy, halogen substituted benzyloxy, halogen substituted phenyl(C₁₋₄)alkyl, halogen substituted aryloxy, or a halogen substituted (C₆₋₁₀)aryl, or Z can also be hydroxy; and R³⁰, R³¹, R³² and R³³ are in each instance independently hydrogen, hydroxy, C₁₋₄ alkoxy, C₁₋₄ alkyl, or hydroxy(C₁₋₄)alkyl;

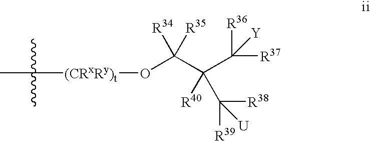

wherein Y is hydrogen, hydroxy, halogen, (C₁₋₄)alkoxy, (C₁₋₄)alkyl, or hydroxy(C₁₋₄)alkyl; U is hydrogen, hydroxy, halogen, halogen substituted benzoyloxy, halogen substituted phenyl(C₁₋₄)alkyl, halogen substituted aryloxy, or halogen substituted (C₆₋₁₀)aryl; and R³⁴, R³⁵, R³⁶, R³⁷, R³⁸, R³⁹ and R⁴⁰ are in each instance independently hydrogen, halogen, hydroxy, (C₁₋₄)alkoxy, (C₁₋₄)alkyl, or hydroxy(C₁₋₄)alkyl;
iii. NR^{†}R^{††}, wherein at least one of R^{†} and R^{††} is (CH₂)_{d}X, where X is halogen, preferably F or ¹⁸F, and d is an integer from 1 to 4; the other of R^{†} and R^{††} is hydrogen, (C₁₋₄)alkyl, halo(C₁₋₄)alkyl, or hydroxy(C₁₋₄)alkyl;
iv. NR^{†}R^{††}-(C₁₋₄)alkyl, wherein at least one of R^{†} and R^{††} is (CH₂)_{d}X, where X is halogen, preferably F or ¹⁸F, and d is an integer from 1 to 4; the other of R^{†} and R^{††} is hydrogen, (C₁₋₁₄)alkyl, halo(C₁₋₄)alkyl, or hydroxy(C₁₋₄)alkyl;
v. halo(C₁₋₄)alkyl; or
vi. an ether (R—O—R) having the following structure:
   [halo(C₁₋₄)alkyl-O—(C₁₋₄)alkyl]-;

R³ is a halogen, radiohalogen, halo(C₁₋₄)alkyl, radiohalo(C₁₋₄)alkyl, Si(C₁₋₄ alkyl)₃ or Sn(alkyl)₃; and

R⁷ and R⁸ are in each instance independently hydrogen, hydroxy, amino, methylamino, dimethylamino, (C₁₋₄)alkoxy, (C₁₋₄)alkyl, or hydroxy(C₁₋₄)alkyl.

Preferred compounds include those where the halogen, in one or more occurrences on the structure, is a radiolabeled halogen. Also preferred are compounds wherein the halogen is ¹²³I, ¹²⁴I, ¹²⁵I, ¹³¹I, Br, ⁷⁶Br, ⁷⁷Br, ⁷⁹Br, F or ¹⁸F. Especially preferred compounds are those that contain ¹⁸F. Compounds containing ¹²³I are also especially preferred.

Useful values of R¹ and R^{1†} are listed above. Preferred values are hydroxy or NR^{a}R^{e}(CH₂)ₚ—, wherein p is an integer from 0 to 5, and R^{a} and R^{b} are independently hydrogen, (C₁₋₄)alkyl or (CH₂)_{d}X, where X is halogen, and d is an integer from 1 to 4 Useful values of p include integers from 0 to 5. Preferably, p is 0, 1 or 2. Most preferably, p is 0 such that R¹ or R^{1†} represents NR^{a}R^{b}. In preferred embodiments, R¹ is hydrogen, and R¹ is either in the meta or para position relative to the respective bridge. A preferred value of R¹ is NR^{a}R^{b}, wherein R^{a} and R^{b} are independently hydrogen or (C₁₋₄)alkyl. In this embodiment, it is preferable that the (C₁₋₄)alkyl is methyl. Preferably one of R^{a} and R^{b} is hydrogen, the other is C₁₋₄ alkyl, such as methyl. Most preferably, both R^{a} and R^{b} are methyl. Another preferred value of R¹ is hydroxy. Also preferred are any prodrug groups that after administration yield a preferred value of R¹. Such prodrug groups are well-known in the art.

Useful values of n include integers from 1 to 6. Preferably, the value of n is from 1 to 4. Most preferably, the value of n is from 1 to 3. It is especially preferred that n is one.

Useful values of R⁷ and R⁸ are in each instance independently hydrogen, hydroxy, amino, methylamino, dimethylamino, (C₁₋₄)alkoxy, (C₁₋₄)alkyl, or hydroxy(C₁₋₄)alkyl. The value of n determines the number of R⁷ and R⁸ group(s) present in the compound. If present more than once in a particular compound, in each instance of R⁷ and R⁸ the value can be different from any other value of R⁷ and R⁸. In preferred embodiments, R⁷ and R⁸ are each hydrogen in every instance.

Useful values of R² include substructures i, i^{†}, ii, ii^{†}, iii, iv, v, and vi, as depicted above. In preferred embodiments of Formula I, R² is either in the meta or para position relative to the respective bridge. Preferably, R² is substructure i or ii. Also preferred are substructures i^{†} and ii^{†}. In these embodiments, useful values of q include integers from one to ten. Preferably, in a compound where R² is i or i^{†}, q is an integer from 1 to 5. Most preferably, q is 1 to 4, especially 3 or 4. In substructure i or i^{†}, useful values of R³⁰, R³¹, R³² and R³³ independently include hydrogen, hydroxy, C₁₋₄ alkoxy, C₁₋₄ alkyl, and hydroxy(C₁₋₄)alkyl. Preferred compounds include those where one or more of R³⁰, R³¹, R³² and R³³ are hydrogen. More preferred compounds include those where each of R³⁰, R³¹, R³² and R³³ is hydrogen.

Useful values of R³ include all those listed above. Preferably R³ is ¹²⁵I, ¹²³I, ¹³¹I, ¹⁸F, ¹⁸F(C₁-C₄)alkyl, ⁷⁶Br, ⁷⁷Br or Sn(alkyl)₃.

In substructure ii or ii^{†}, useful values of Y, U and R³⁴, R³⁵, R³⁶, R³⁷, R³⁸, R³⁹ and R⁴⁰ are described above. Preferred compounds include those where U is hydroxy.

Especially preferred compounds of Formula I include the following:

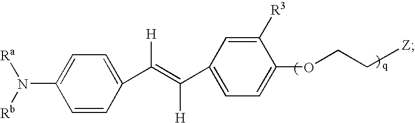

wherein R^{a} and R^{b} are independently (C₁₋₄)alkyl and q is an integer from 1 to 4 and R³ is preferably ¹²³I or ¹⁸F;

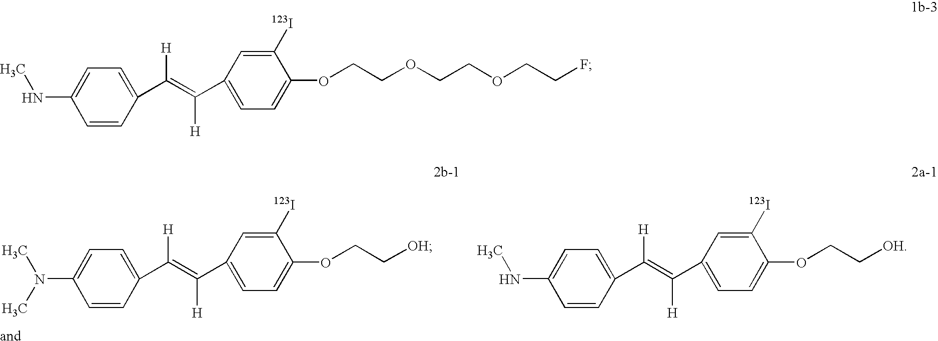

Other preferred compounds of Formula III, when R² is ii, include:

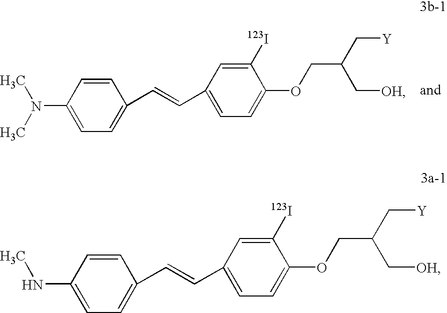

wherein Y is hydrogen or F.

Compounds of Formula I when R² is i, or i^{†} when t is 0, include hydroxy ethers such as:

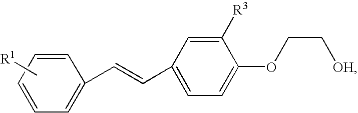

wherein R¹ and R³ are as described above under Formula I.

In all embodiments of Formula I containing —(CR^{x}R^{y})ₜ where t is other than zero, the compounds have the following general structure wherein there is at least one carbon-carbon bond between a substituent and the nitrogen-containing ring:

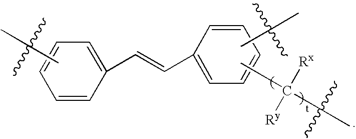

The compounds of the present invention can also contain a radioactive isotope of carbon as the radiolabel. This refers to a compound that comprises one or more radioactive carbon atoms, preferably ¹¹C, with a specific activity above that of the background level for that atom. It is well known, in this respect, that naturally occurring elements are present in the form of varying isotopes, some of which are radioactive isotopes. The radioactivity of the naturally occurring elements is a result of the natural distribution or abundance of these isotopes, and is commonly referred to as a background level. The carbon labeled compounds of the present invention have a specific activity that is higher than the natural abundance, and therefore above the background level. The composition claimed herein comprising a carbon-labeled compound(s) of the present invention will have an amount of the compound such that the composition can be used for tracing, imaging, radiotherapy, and the like.

The compounds of Formula I may also be solvated, especially hydrated. Hydration may occur during manufacturing of the compounds or compositions comprising the compounds, or the hydration may occur over time due to the hygroscopic nature of the compounds. In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

When any variable occurs more than one time in any constituent or in Formula I its definition on each occurrence is independent of its definition at every other occurrence. Also combinations of substituents and/or variables are permissible only if such combinations result in stable compounds.

The term “alkyl” as employed herein by itself or as part of another group refers to both straight and branched chain radicals of up to 8 carbons, preferably 6 carbons, more preferably 4 carbons, such as methyl, ethyl, propyl, isopropyl, butyl, t-butyl, and isobutyl.

The term “alkoxy” is used herein to mean a straight or branched chain alkyl radical, as defined above, unless the chain length is limited thereto, bonded to an oxygen atom, including, but not limited to, methoxy, ethoxy, n-propoxy, isopropoxy, and the like. Preferably the alkoxy chain is 1 to 6 carbon atoms in length, more preferably 1-4 carbon atoms in length.

The term “monoalkylamine” as employed herein by itself or as part of another group refers to an amino group which is substituted with one alkyl group as defined above.

The term “dialkylamine” as employed herein by itself or as part of another group refers to an amino group which is substituted with two alkyl groups as defined above.

The term “halo” or “halogen” employed herein by itself or as part of another group refers to chlorine, bromine, fluorine or iodine and their isotopes. The term “radiohalogen” refers specifically to radioactive halogen isotopes.

The term “haloalkyl” as employed herein refers to any of the above alkyl groups substituted by one or more chlorine, bromine, fluorine or iodine with fluorine and chlorine being preferred, such as chloromethyl, iodomethyl, trifluoromethyl, 2,2,2-trifluoroethyl, and 2-chloroethyl.

The term “alkylthio” as employed herein by itself or as part of another group refers to a thioether of the structure: R—S, wherein R is a C₁₋₄ alkyl as defined above.

The term “alkylsulfonyl” as employed herein by itself or as part of another group refers to a sulfone of the structure: R—SO₂, wherein R is a C₁₋₄ alkyl as defined above.

The term “aryl” as employed herein by itself or as part of another group refers to monocyclic or bicyclic aromatic groups containing from 6 to 12 carbons in the ring portion, preferably 6-10 carbons in the ring portion, such as phenyl, naphthyl or tetrahydronaphthyl. Preferable values under the scope of C₆₋₁₀ aryl include phenyl, naphthyl or tetrahydronaphthyl. Preferable values of under the scope of heteroaryl include thienyl, furyl, pyranyl, pyrrolyl, pyridinyl, indolyl, and imidazolyl. Preferable values under the scope of heterocycle include piperidinyl, pyrrolidinyl, and morpholinyl.

A preferred embodiment of a C₆₋₁₀ aryl, heteroaryl, heterocycle, heterocycle(C₁₋₄)alkyl or C₃₋₆ cycloalkyl, contains a ring substituted with one of the following: C₁₋₄ alkylthio, C₁₋₄ alkyl sulfonyl, methoxy, hydroxy, dimethylamino or methylamino.

The term “heterocycle” or “heterocyclic ring”, as used herein except where noted, represents a stable 5- to 7-membered mono-heterocyclic ring system which may be saturated or unsaturated, and which consists of carbon atoms and from one to three heteroatoms selected from the group consisting of N, O, and S, and wherein the nitrogen and sulfur heteroatom may optionally be oxidized. Especially useful are rings contain one nitrogen combined with one oxygen or sulfur, or two nitrogen heteroatoms. Examples of such heterocyclic groups include piperidinyl, pyrrolyl, pyrrolidinyl, imidazolyl, imidazinyl, imidazolidinyl, pyridyl, pyrazinyl, pyrimidinyl, oxazolyl, oxazolidinyl, isoxazolyl, isoxazolidinyl, thiazolyl, thiazolidinyl, isothiazolyl, homopiperidinyl, homopiperazinyl, pyridazinyl, pyrazolyl, and pyrazolidinyl, most preferably thiamorpholinyl, piperazinyl, and morpholinyl.

The term “heteroatom” is used herein to mean an oxygen atom (“O”), a sulfur atom (“S”) or a nitrogen atom (“N”). It will be recognized that when the heteroatom is nitrogen, it may form an NRR moiety, wherein the R groups independently from one another may be hydrogen or C₁₋₄ alkyl, C₂₋₄ aminoalkyl, C₁₋₄ halo alkyl, halo benzyl, or R¹ and R² are taken together to form a 5- to 7-member heterocyclic ring optionally having O, S or NR^{c} in said ring, where R^{c} is hydrogen or C₁₋₄ alkyl.

The term “heteroaryl” as employed herein refers to groups having 5 to 14 ring atoms; 6, or 14 μl electrons shared in a cyclic array; and containing carbon atoms and 1, 2, 3 or 4 oxygen, nitrogen or sulfur heteroatoms (where examples of heteroaryl groups are: thienyl, benzo[b]thienyl, naphtho[2,3-b]thienyl, thianthrenyl, furyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4aH-carbazolyl, carbazolyl, α, β, or γ-carbolinyl, phenanthridinyl, acridinyl, perimidinyl, phenanthrolinyl, phenazinyl, isothiazolyl, phenothiazinyl, isoxazolyl, furazanyl and phenoxazinyl groups).

The term “aralkyl” or “arylalkyl” as employed herein by itself or as part of another group refers to C₁₋₆ alkyl groups as discussed above having an aryl substituent, such as benzyl, phenylethyl or 2-naphthylmethyl.

Another aspect of this invention is related to methods of preparing compounds of Formula I. Synthetic routes for preparing compounds of the invention include the following syntheses.

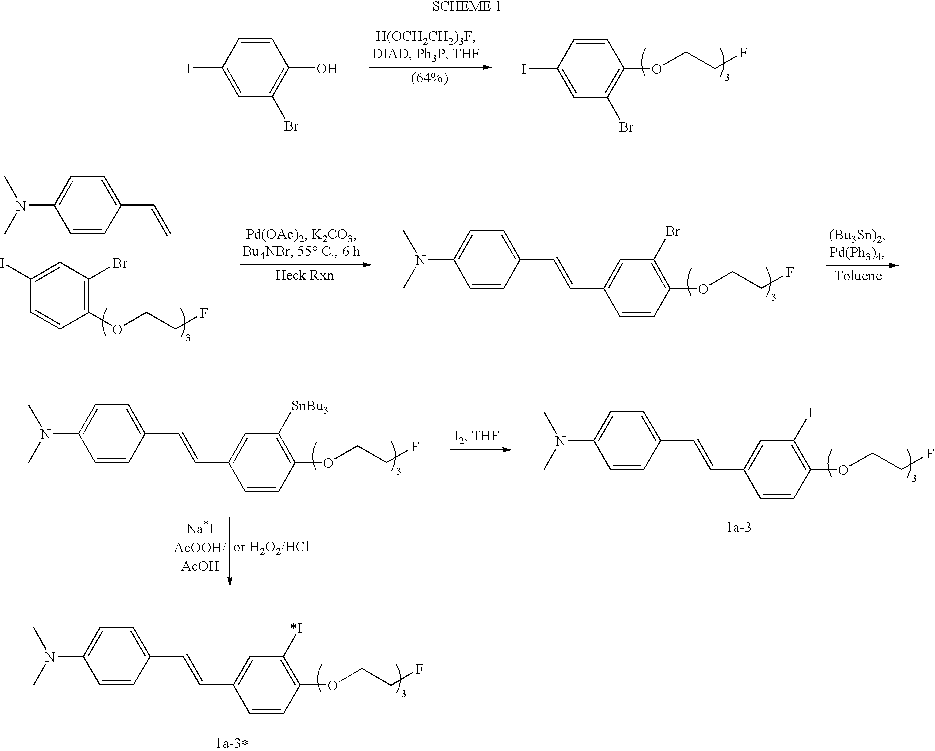

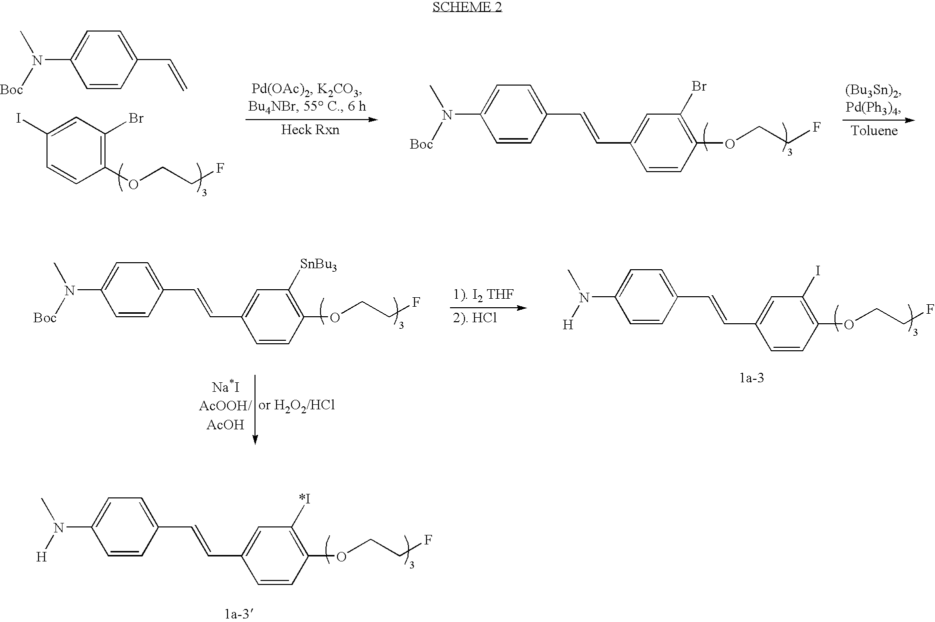

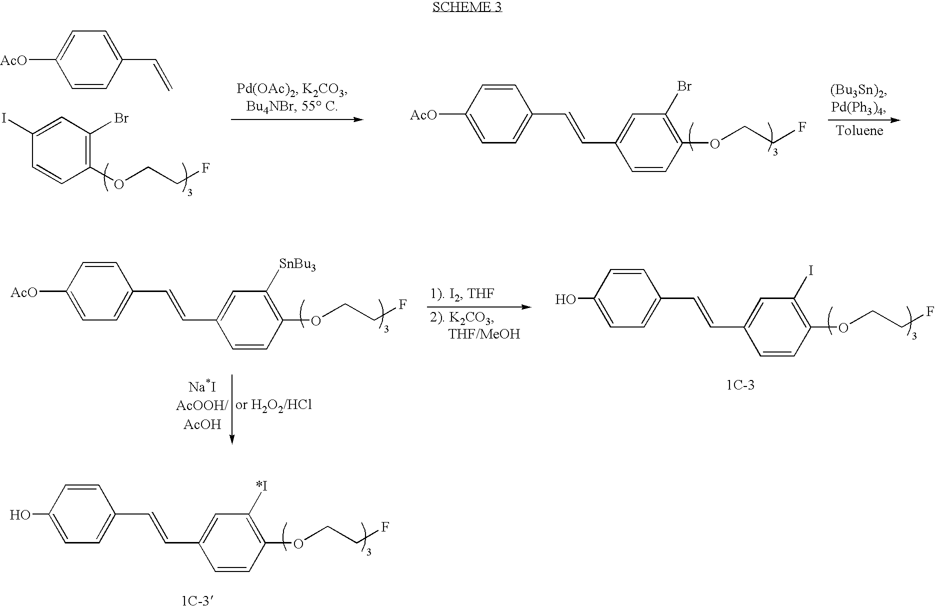

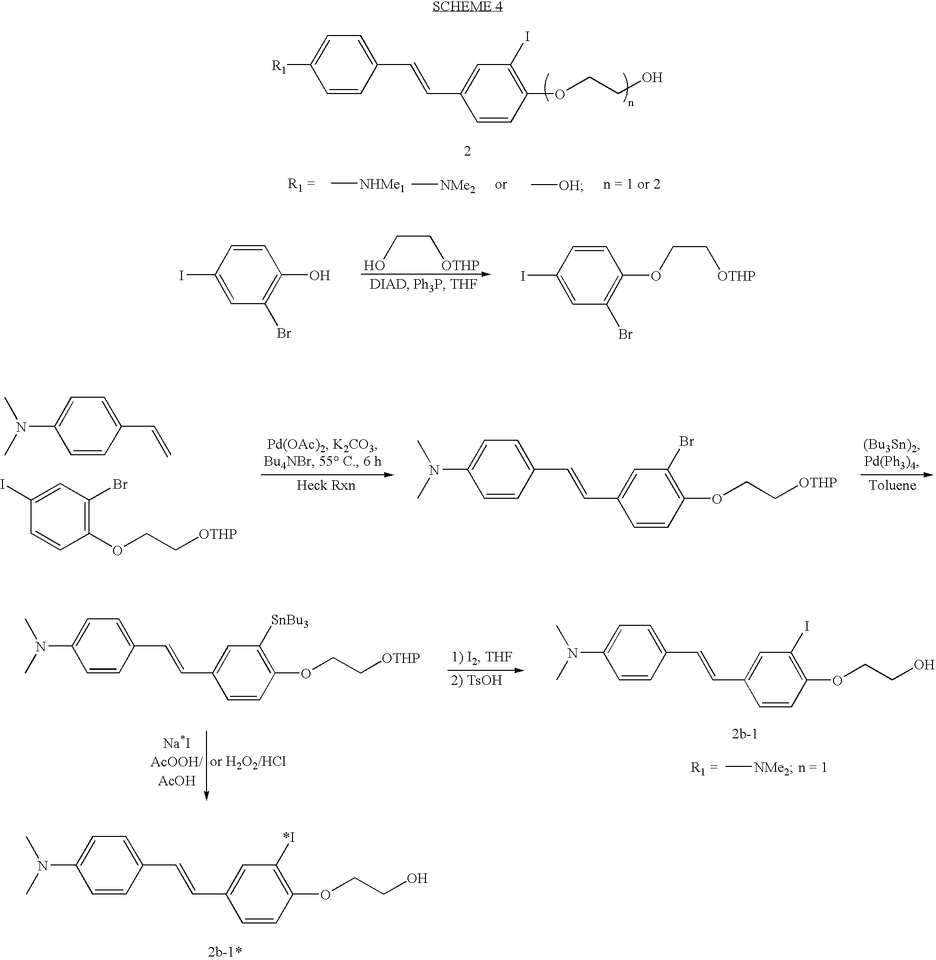

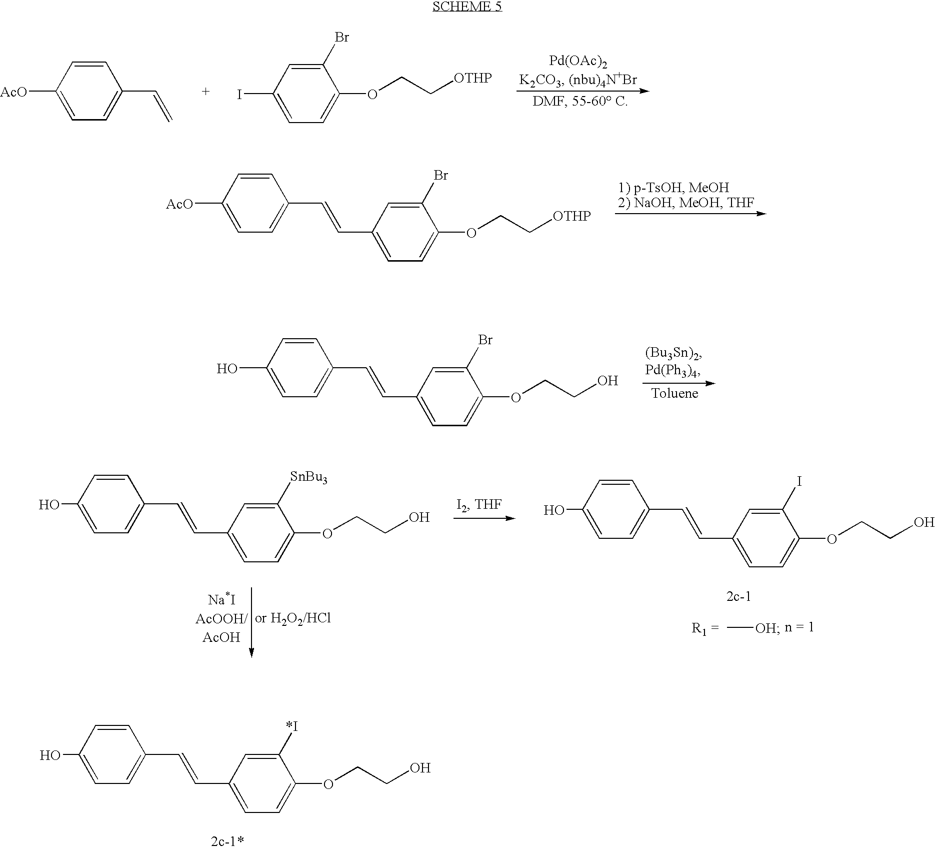

When the compounds of this invention are to be used as imaging agents, they must be labeled with suitable radioactive halogen isotopes. Although ¹²⁵I-isotopes are useful for laboratory testing, they will generally not be useful for actual diagnostic purposes because of the relatively long half-life (60 days) and low gamma-emission (30-65 Kev) of ¹²⁵I. The isotope ¹²³, has a half life of thirteen hours and gamma energy of 159 KeV, and it is therefore expected that labeling of ligands to be used for diagnostic purposes would be with this isotope. Other isotopes which may be used include 131, (half life of 2 hours). Suitable bromine isotopes include ⁷⁷Br and ⁷⁶Br.

The radiohalogenated compounds of this invention lend themselves easily to formation from materials which could be provided to users in kits. Kits for forming the imaging agents can contain, for example, a vial containing a physiologically suitable solution of an intermediate of Formula I in a concentration and at a pH suitable for optimal complexing conditions. The user would add to the vial an appropriate quantity of the radioisotope, e.g., Na ¹²³, and an oxidant, such as hydrogen peroxide. The resulting labeled ligand may then be administered intravenously to a patient, and receptors in the brain imaged by means of measuring the gamma ray or photo emissions therefrom.

When desired, the radioactive diagnostic agent may contain any additive such as pH controlling agents (e.g., acids, bases, buffers), stabilizers (e.g., ascorbic acid) or isotonizing agents (e.g., sodium chloride).

The term “pharmaceutically acceptable salt” as used herein refers to those carboxylate salts or acid addition salts of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively nontoxic, inorganic and organic acid addition salts of compounds of the present invention. Also included are those salts derived from non-toxic organic acids such as aliphatic mono and dicarboxylic acids, for example acetic acid, phenyl-substituted alkanoic acids, hydroxy alkanoic and alkanedioic acids, aromatic acids, and aliphatic and aromatic sulfonic acids. These salts can be prepared in situ during the final isolation and purification of the compounds or by separately reacting the purified compound in its free base form with a suitable organic or inorganic acid and isolating the salt thus formed. Further representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactiobionate and laurylsulphonate salts, propionate, pivalate, cyclamate, isethionate, and the like. These may include cations based on the alkali and alkaline earth metals, such as sodium, lithium, potassium, calcium, magnesium, and the like, as well as, nontoxic ammonium, quaternary ammonium and amine cations including, but not limited to ammonium, tetramethylammonium, tetraethylammonium, methylamine, dimethylamine, trimethylamine, triethylamine, ethylamine, and the like. (See, for example, Berge S. M., et al., Pharmaceutical Salts, J. Pharm. Sci. 66:1-19 (1977) which is incorporated herein by reference.)

In the first step of the present method of imaging, a labeled compound of Formula I is introduced into a tissue or a patient in a detectable quantity. The compound is typically part of a pharmaceutical composition and is administered to the tissue or the patient by methods well known to those skilled in the art.

The administration of the labeled compound to a patient can be by a general or local administration route. For example, the compound can be administered either orally, rectally, parenterally (intravenous, by intramuscularly or subcutaneously), intracisternally, intravaginally, intraperitoneally, intravesically, locally (powders, ointments or drops), or as a buccal or nasal spray. The labeled compound may be administered to the patient such that it is delivered throughout the body. Alternatively, the labeled compound can be administered to a specific organ or tissue of interest. For example, it is desirable to locate and quantitate amyloid deposits in the brain in order to diagnose or track the progress of Alzheimer's disease in a patient. One of the most desirable characteristics of an in vivo imaging agent of the brain is the ability to cross the intact blood-brain barrier after a bolus iv injection.

In a preferred embodiment of the invention, the labeled compound is introduced into a patient in a detectable quantity and after sufficient time has passed for the compound to become associated with amyloid deposits, the labeled compound is detected noninvasively inside the patient. In another embodiment of the invention, a radiolabeled compound of Formula I is introduced into a patient, sufficient time is allowed for the compound to become associated with amyloid deposits, and then a sample of tissue from the patient is removed and the labeled compound in the tissue is detected apart from the patient. In a third embodiment of the invention, a tissue sample is removed from a patient and a labeled compound of Formula I is introduced into the tissue sample. After a sufficient amount of time for the compound to become bound to amyloid deposits, the compound is detected.

The term “tissue” means a part of a patient's body. Examples of tissues include the brain, heart, liver, blood vessels, and arteries. A detectable quantity is a quantity of labeled compound necessary to be detected by the detection method chosen. The amount of a labeled compound to be introduced into a patient in order to provide for detection can readily be determined by those skilled in the art. For example, increasing amounts of the labeled compound can be given to a patient until the compound is detected by the detection method of choice. A label is introduced into the compounds to provide for detection of the compounds.

The term “patient” means humans and other animals. Those skilled in the art are also familiar with determining the amount of time sufficient for a compound to become associated with amyloid deposits. The amount of time necessary can easily be determined by introducing a detectable amount of a labeled compound of Formula I into a patient and then detecting the labeled compound at various times after administration.

The term “associated” means a chemical interaction between the labeled compound and the amyloid deposit. Examples of associations include covalent bonds, ionic bonds, hydrophilic-hydrophilic interactions, hydrophobic-hydrophobic interactions, and complexes.

Those skilled in the art are familiar with the various ways to detect labeled compounds. For example, magnetic resonance imaging (MRI), positron emission tomography (PET), or single photon emission computed tomography (SPECT) can be used to detect radiolabeled compounds. The label that is introduced into the compound will depend on the detection method desired. For example, if PET is selected as a detection method, the compound must possess a positron-emitting atom, such as ¹¹C or ¹⁸F.

The radioactive diagnostic agent should have sufficient radioactivity and radioactivity concentration which can assure reliable diagnosis. For instance, in case of the radioactive metal being technetium-99 m, it may be included usually in an amount of 0.1 to 50 mCi in about 0.5 to 5.0 ml at the time of administration.

The imaging of amyloid deposits can also be carried out quantitatively so that the amount of amyloid deposits can be determined.

Another aspect of the invention is a method of inhibiting amyloid plaque aggregation. The present invention also provides a method of inhibiting the aggregation of amyloid proteins to form amyloid deposits, by administering to a patient an amyloid inhibiting amount of a compound of the above Formula I.

Those skilled in the art are readily able to determine an amyloid inhibiting amount by simply administering a compound of Formula I to a patient in increasing amounts until the growth of amyloid deposits is decreased or stopped. The rate of growth can be assessed using imaging as described above or by taking a tissue sample from a patient and observing the amyloid deposits therein. The compounds of the present invention can be administered to a patient at dosage levels in the range of about 0.1 to about 1,000 mg per day. For a normal human adult having a body weight of about 70 kg, a dosage in the range of about 0.01 to about 100 mg per kilogram of body weight per day is sufficient. The specific dosage used, however, can vary. For example, the dosage can depend on a number of factors including the requirements of the patient, the severity of the condition being treated, and the pharmacological activity of the compound being used. The determination of optimum dosages for a particular patient is well known to those skilled in the art.

The following examples are illustrative, but not limiting, of the method and compositions of the present invention. Other suitable modifications and adaptations of the variety of conditions and parameters normally encountered and obvious to those skilled in the art are within the spirit and scope of the invention.

### EXAMPLES

### Example 1

### (E)-2-(3-iodo-5-(4-(methylamino)styrl)phenoxy)ethanol

¹²³I-15: (E)-2-(2-[¹²³I]iodo-4-(4-(methylamino)styryl)phenoxy)ethanol

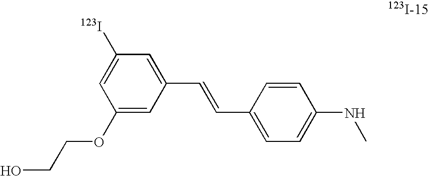

151 (E)-2-(3-iodo-5-(4-(methylamino)styryl)phenoxy)ethanol, cold reference standard for ¹²³I-15

### SUMMARY

TABLE 1
Pharmacological properties of Compound 15

| | | | | | Biodistribution in | |
|---|---|---|---|---|---|---|
| | | | | | Mice | |
| | | | Specificity | | (% dose/g in brain) | |
| | Binding | Binding Affinity | for Plaques | | 2 | 60 |
| | Target | (Ki, nM) | (section labeling) | Selectivity | min | min |
| 15 | Amyloid Plaque | 9.4 ± 1.8 | [symbol] | No high affinity binding to any CNS receptors | 3.40 ± 0.3 | 0.43 ± 0.05 |

Compound 15 shows high affinity and specific binding to amyloid plaques, as demonstrated by competitive binding studies using the known amyloid binding agent ¹²⁵I-IMPY (6-iodo-2-(4′-dimethylamino-)phenyl-imidazo[1,2-a]pyridine). In these experiments Compound 15 showed a Ki of 9.4±1.8 nM, comparable to other experimental amyloid imaging agents. ¹²³I-15, when applied at tracer concentrations, specifically labeled Aβ plaques in sections from patients with pathologically confirmed AD.

As is shown in Table 1, Compound 15 exhibited high affinity specific binding to amyloid plaques in competitive binding studies using the known amyloid binding agent 125I-IMPY. In these experiments, Compound 15 showed a Kᵢ of 9.4±1.8 nM in competitive binding with ¹²⁵I-IMPY, comparable to other experimental amyloid imaging agents. In-vitro autoradiography studies also confirm that at tracer concentrations ¹²³I-15 labeled Aβ plaques in post-mortem brain sections from AD subjects.

In mouse in-vivo experiments ¹²³I-15 showed appropriate biodistribution for a brain imaging agent. When administered i.v. to male mice, ¹²³I-15 entered the brain quickly and reached a peak brain concentration of 3.4% dose/g within 2 min post administration and fell to 0.43% dose/g within 60 min. An estimate of human dosimetry, based on extrapolation from the mouse data, suggests that the dose-limiting organs (assuming thyroid blocking) will be the intestines. At the proposed 5 mCi human dose, the lower large intestine is estimated to receive approximately 4.08 rem, while the estimated human effective dose equivalent (ED) of approximately 1.1 rem is comparable to that for recommended doses of other ¹²³I-labeled SPECT imaging radiopharmaceuticals such as ¹²³I-MIBG, ¹²³-IMPY, and ¹²³I-βCIT.

### Binding Affinity of Compound 15 in AD Brain Homogenates, as Measured by Inhibition of ¹²⁵I-IMPY Binding

It has been previously demonstrated that ¹²⁵I-IMPY binds with high affinity to brain homogenates from patients with AD. Moreover, the binding of IMPY appears to be specific for cortical vs. cerebellar tissue from patients with AD, is largely absent in cortex homogenates from elderly control brains, and is fully displaceable by the presumed amyloid imaging agent PIB. Autoradiographic studies in slices from these same brains indicates that ¹²⁵I-IMPY labels amyloid plaques and co-localizes with Thioflavin-S staining. Thus, inhibition of ¹²⁵I-IMPY binding in human AD brain homogenates was taken as an assay for the potential of test compounds to bind to amyloid plaques.

Postmortem brain tissue was obtained and neuropathological diagnosis was confirmed in accordance with the NIA-Reagan Institute Consensus Group criteria. Homogenates were then prepared from dissected gray matter, pooled in phosphate buffered saline and aliquoted into 1-ml portions (100 mg wet tissue/ml), which could be stored at −70° C. for 3-6 months without loss of binding signal.

For the binding assays brain homogenates were incubated with ¹²⁵I-IMPY (0.04-0.06 nM diluted in phosphate buffered saline (PBS)) and test compound (10⁻⁵-10⁻¹⁰ M diluted in PBS containing 0.1% bovine serum albumin (BSA)). Nonspecific binding was defined in the presence of IMPY (600 nM). The bound and free radioactivity was separated by vacuum filtration followed by 2×3 ml washes of PBS. Filters containing the bound ¹²⁵I ligand were assayed in a gamma counter.

Compound 15 potently inhibited ¹²⁵I-IMPY binding in this assay, with a Ki=9.4±1.8 nM. This is comparable to the Ki values published for other presumed amyloid imaging agents that have been tested in humans (Table 2).

TABLE 2
Binding affinity of Compound 15 and other amyloid plaque ligands to
AD brain homogenates (Ki vs. ¹²⁵I-IMPY)

| | 15 | PIB | FDDNP | IMPY | SB-13 |
|---|---|---|---|---|---|
| Ki (nM) ± SD | 9.4 ± 1.8 | 2.8 ± 0.5 | 239 | 5.0 ± 0.4 | 1.2 ± 0.2 |

PIB = Pittsburgh Compound B, N-methyl[11C]2-(4′-methylaminophenyl-6-hydroxybenzathiazole
FDDNP = 2-(1-{6-[(2-[¹⁸F]fluoroethyl)(methyl)amino]-2-naphthyl}ethylidene)malononitrile
IMPY = 6-iodo-2-(4′-dimethylamino-)phenyl-imidazo[1,2]pyridine
SB-13 - 4-N-methylamino-4′-hydroxystilbene

### Film Autoradiography

Brain sections from a patient with AD were frozen in powdered dry ice and cut into 20 μm thick sections. The sections were incubated with ¹²³I-15 (200,000-250,000 cpm/200 μl) for 1 hour at room temperature. The sections were then dipped in saturated Li₂CO₃ in 40% EtOH (2×2 min. washes) and washed with 40% EtOH (1×2 min. wash) followed by rinsing with water for 30s. After drying, the ¹²³I-labeled sections were exposed to Kodak MR film overnight.

Autoradiographic visualization of amyloid plaques was observed in grey matter of the postmortem AD brains.

Binding at CNS and Cardiovascular Receptors

A panel of 46 CNS and Cardiovascular receptor binding sites, including adenosine, adrenergic, benzodiazepine, bombesin, dopamine, GABA, melatonin, muscarinic, neurotensin, nicotininc acetylcholine, opiate, potassium channel, serotonin, sigma, and several transporter subtypes, was tested for binding of Compound 15. The aim of the study was to determine the inhibition of binding of known ligands of these receptors when incubated with Compound 15 (1 μM). Inhibition of binding in excess of 50% by Compound 15 was considered potentially significant. No significant binding was observed at any of the 46 CNS and CV receptor sites.

### Organ Distribution in Normal Mice

While under isoflurane anesthesia, 0.15 ml of a 0.9% saline solution containing ¹²³I-15 (5-10 μCi) was injected directly into the tail vein of ICR mice (22-25 g, male). The mice (n=3 for each time point) were sacrificed, the organs of interest were removed, weighed, and assayed with an automatic gamma counter. The percentage dose per organ was calculated by a comparison of the tissue counts to suitably diluted aliquots of the injected material. Total activities of blood were calculated under the assumption that they were 7% of the total body weight. The % dose/g of samples was calculated by comparing the sample counts with the count of the diluted initial dose. The results (Table 4) suggest ¹²³I-15 readily penetrates and is rapidly cleared from the brain of normal mice. Similar results were obtained in female mice.

TABLE 3
Biodistribution of ¹²³I-15 after an i.v. injection in normal male mice
(average of 3 mice ± SD) % dose/organ and % dose/g
of blood, muscle, skin and bone were calculated under the assumption
that these tissues represent 7%, 40%, 15% and 14% of body mass respectively.

| % dose/organ | | | | |
|---|---|---|---|---|
| Organ | 2 min | 60 min | 120 min | 180 min |
| Blood | 12.82 ± 1.41 | 5.13 ± 0.38 | 6.96 ± 1.02 | 4.39 ± 1.95 |
| Heart | 0.94 ± 0.11 | 0.17 ± 0.03 | 0.21 ± 0.06 | 0.14 ± 0.04 |
| Muscle | 22.33 ± 2.32 | 5.77 ± 0.69 | 8.68 ± 4.91 | 5.10 ± 2.44 |
| Lung | 2.10 ± 0.24 | 0.57 ± 0.11 | 0.86 ± 0.54 | 0.38 ± 0.15 |
| Kidney | 4.60 ± 0.21 | 2.83 ± 0.54 | 1.69 ± 0.30 | 1.06 ± 0.19 |
| Spleen | 1.09 ± 0.36 | 0.68 ± 0.15 | 0.60 ± 0.13 | 0.39 ± 0.15 |
| Liver | 24.50 ± 2.76 | 16.76 ± 1.76 | 10.54 ± 0.32 | 7.72 ± 0.66 |
| Skin | 3.17 ± 1.08 | 5.30 ± 0.36 | 5.31 ± 1.39 | 4.49 ± 2.06 |
| Brain | 1.49 ± 0.12 | 0.21 ± 0.01 | 0.11 ± 0.03 | 0.08 ± 0.03 |
| Bone | 6.90 ± 0.68 | 3.14 ± 1.45 | 4.53 ± 1.65 | 3.35 ± 1.21 |
| Thyroid | 0.04 ± 0.01 | 0.49 ± 0.39 | 1.78 ± 1.17 | 2.39 ± 0.89 |
| Pancreas | 0.72 ± 0.22 | 0.25 ± 0.01 | 0.32 ± 0.07 | 0.22 ± 0.11 |
| Stomach | 0.84 ± 0.20 | 8.33 ± 2.10 | 11.06 ± 2.99 | 7.55 ± 5.20 |
| Intestine | 5.85 ± 0.57 | 33.98 ± 6.67 | 28.80 ± 1.55 | 42.64 ± 15.13 |
| Urogenital | 0.44 ± 0.16 | 2.66 ± 1.76 | 1.45 ± 0.52 | 1.13 ± 0.43 |
| Testes | 0.16 ± 0.01 | 0.21 ± 0.04 | 0.21 ± 0.09 | 0.17 ± 0.07 |
| Tail | 10.11 ± 2.69 | 2.39 ± 0.40 | 1.10 ± 0.02 | 1.30 ± 0.66 |
| Fat | 0.58 ± 0.53 | 0.50 ± 0.08 | 0.41 ± 0.02 | 0.32 ± 0.10 |
| Carcass | 27.00 ± 1.10 | 14.45 ± 2.71 | 16.93 ± 3.11 | 11.28 ± 3.77 |
| % total | 86.65 ± 1.34 | 87.53 ± 4.57 | 79.41 ± 5.56 | 79.46 ± 5.15 |
| counted | | | | |

Table 3b: Biodistribution of ¹²³I-15 after an i.v. injection in normal male mice
(average of 3 mice ± SD) % dose/organ and % dose/g
of blood, muscle, skin and bone were calculated under the assumption
that these tissues represent 7%, 40%, 15% and 14% of body mass respectively.

| % dose/g | | | | |
|---|---|---|---|---|
| Organ | 2 min | 60 min | 120 min | 180 min |
| Blood | 6.30 ± 0.74 | 2.67 ± 0.43 | 3.25 ± 0.51 | 2.20 ± 1.15 |
| Heart | 7.36 ± 1.12 | 1.41 ± 0.03 | 1.32 ± 0.39 | 1.07 ± 0.44 |
| Muscle | 1.93 ± 0.33 | 0.52 ± 0.04 | 0.72 ± 0.45 | 0.45 ± 0.25 |
| Lung | 10.50 ± 1.40 | 2.90 ± 0.52 | 6.59 ± 4.06 | 2.38 ± 1.09 |
| Kidney | 9.40 ± 0.61 | 5.74 ± 0.80 | 3.59 ± 2.00 | 2.14 ± 0.15 |
| Spleen | 15.24 ± 2.35 | 6.90 ± 2.93 | 3.74 ± 0.45 | 4.43 ± 2.22 |
| Liver | 19.46 ± 1.22 | 12.97 ± 3.82 | 8.18 ± 3.78 | 5.94 ± 0.67 |
| Skin | 0.72 ± 0.19 | 1.28 ± 0.15 | 1.15 ± 0.25 | 1.05 ± 0.56 |
| Brain | 3.40 ± 0.31 | 0.43 ± 0.05 | 0.24 ± 0.03 | 0.16 ± 0.07 |
| Bone | 1.70 ± 0.28 | 0.81 ± 0.39 | 1.04 ± 0.34 | 0.84 ± 0.36 |
| Thyroid | 5.34 ± 2.89 | 23.53 ± 14.83 | 173.55 ± 120.43 | 240.75 ± 114.25 |
| Pancreas | 3.66 ± 0.75 | 1.31 ± 0.14 | 1.51 ± 0.34 | 1.16 ± 0.56 |
| Stomach | 1.62 ± 0.05 | 12.12 ± 3.91 | 25.47 ± 7.73 | 15.56 ± 9.75 |
| Intestine | 2.21 ± 0.20 | 12.45 ± 2.52 | 11.46 ± 0.84 | 18.06 ± 4.48 |
| Urogenital | 0.94 ± 0.09 | 5.78 ± 4.12 | 3.25 ± 1.01 | 2.64 ± 1.22 |
| Testes | 0.84 ± 0.10 | 0.88 ± 0.17 | 0.84 ± 0.22 | 0.96 ± 0.76 |
| Tail | 13.93 ± 4.26 | 3.15 ± 0.67 | 1.40 ± 0.06 | 1.65 ± 0.78 |
| Fat | 1.01 ± 0.67 | 1.57 ± 0.23 | 1.06 ± 0.12 | 0.71 ± 0.16 |
| Carcass | 1.48 ± 0.08 | 0.86 ± 0.16 | 0.87 ± 0.10 | 0.63 ± 0.26 |

### Partition Coefficient

Partition coefficients were measured by mixing ¹²³I-15 with 3 g each of 1-octanol and buffer (0.1 M phosphate, pH 7.4) in a test tube. The test tube was vortexed for 3 min at room temperature, followed by centrifugation for 5 min. Two weighed samples (0.5 g each) from the 1-octanol and the buffer layer were counted in a well counter. The partition coefficient was determined by calculating the ratio of cpm/g of 1-octanol to that of the buffer. Samples from the 1-octanol layer were repartitioned until consistent partitions of coefficient values were obtained from the third partition. The measurement was done in triplicate and repeated three times. The partition coefficient for ¹²³I-15 was determined to be 249 (logP=2.39).

### Example 2

### (E)-4-(2-(6-(2-(2-(2-fluoroethoxy)ethoxy)ethoxy)phenyl)-vinyl)-N-methylbenzamine and (E)-4-(4-(2-(2-(2-fluoroethoxy)ethoxy)ethoxy)-3-iodostyryl)-N-methylbenzenamine

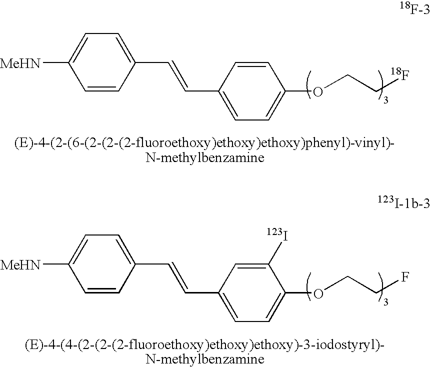

### SUMMARY

Compound 1b-3 showed high affinity and specific binding to amyloid plaques, as demonstrated by competitive binding studies using the known amyloid binding agent ¹²⁵I-IMPY. In those experiments Compound 1b-3 showed a Kᵢ of 9.0±1.8 nM, comparable to Compound 3 and other experimental amyloid imaging agents. ¹²³I-1b-3, when applied at tracer concentrations, specifically labeled Aβ plaques in sections from patients with pathologically confirmed AD.

In both rodent and primate in vivo experiments ¹²³I-1b-3 showed appropriate biodistribution for a brain imaging agent. When administered i.v. to normal male mice, ¹²³I-1b-3 entered the brain and reached a peak brain concentration of 2.0% dose/g within 2 min and fell to 0.51% dose/g within 60 min. An estimate of human dosimetry, based on extrapolation from the mouse data, suggests that the dose-limiting organs (assuming thyroid blocking) will be the intestines. At the proposed 5 mCi human dose the lower large intestine, and the upper large intestine are estimated to receive approximately 4.04 rem and 3.55 rem of exposure, respectively. The estimated human effective dose (ED) of 1.1 rem is comparable to that for recommended doses of other ¹²³I-labeled SPECT brain imaging radiopharmaceuticals such as ¹²³I-MIBG, ¹²³I-IMPY, and ¹²³I-βCIT.

TABLE 4
Pharmacological properties of Compounds 1b-3 and 3

| | | | | | Biodistribution in | |
|---|---|---|---|---|---|---|
| | | | | | Mice | |
| | | | Specificity | | (% dose/g in brain) | |
| | Binding | Binding Affinity | for Plaques | | 2 | 60 |
| | Target | (Ki, nM) | (section labeling) | Selectivity | min | min |
| 1b-3 | Amyloid Plaque | 9.0 ± 0.8 | [symbol] | No high affinity binding to any CNS receptors | 2.0 ± 0.4 | 0.51 ± 0.09 |
| 3 | Amyloid Plaque | 6.7 ± 0.3 | [symbol] | No high affinity binding to any CNS receptors | 4.8 ± 0.2 | 1.5 ± 0.2 |

### Binding Affinity of Compound 1b-3 in Ad Brain Homogenates, as Measured by Inhibition of ¹²⁵I-IMPY Binding

Postmortem brain tissue was obtained and neuropathological diagnosis was confirmed in accordance with the NIA-Reagan Institute Consensus Group criteria. Homogenates were then prepared from dissected gray matter, pooled in phosphate buffered saline and aliquoted into 1-ml portions (100 mg wet tissue/ml), which could be stored at −70° C. for 3-6 months without loss of binding signal.

For the binding assays brain homogenates were incubated with ¹²⁵I-IMPY (0.04-0.06 nM diluted in PBS) and test compound (10⁻⁵-10⁻¹⁰ M diluted in PBS containing 0.1% BSA). Nonspecific binding was defined in the presence of IMPY (600 nM). The bound and free radioactivity was separated by vacuum filtration followed by 2×3 ml washes of PBS. Filters containing the bound 125, ligand were assayed in a gamma counter.

Compound 1b-3 potently inhibited ¹²⁵I-IMPY binding in this assay, with a Kᵢ=9.0±1.8 nM. This is comparable to the Kᵢ values published for other presumed amyloid imaging agents that have been tested in humans (Table 5).

TABLE 5
Binding affinity of Compound 1b-3 and other amyloid plaque ligands to AD brain
homogenates (Ki vs. ¹²⁵I-IMPY)

| | 1b-3 | 3 | PIB | FDDNP | IMPY | SB-13 |
|---|---|---|---|---|---|---|
| Ki (nM) ± SD | 9.0 ± 1.8 | 6.7 ± 0.3 | 2.8 ± 0.5 | 239 | 5.0 ± 0.4 | 1.2 ± 0.2 |

PIB = Pittsburgh Compound B, N-methyl[11C]2-(4′-methylaminophenyl-6-hydroxybenzathiazole
FDDNP = 2-(1-{6-[(2-[¹⁸F]fluoroethyl)(methyl)amino]-2-naphthyl}ethylidene)malononitrile
IMPY = 6-iodo-2-(4′-dimethylamino-)phenyl-imidazo[1,2]pyridine
SB-13 - 4-N-methylamino-4′-hydroxystilbene

### Film Autoradiography

Brains sections from patients with AD were frozen in powdered dry ice and cut into 20 μm thick sections. The sections were incubated with ¹²³I-1b-3 (200,000-250,000 cpm/200 μl) for 1 hour at room temperature. The sections were then dipped in saturated Li₂CO₃ in 40% EtOH (2×2 min. washes) and washed with 40% EtOH (1×2 min. wash) followed by rinsing with water for 30 s. After drying, the ¹²³I-labeled sections were exposed to Kodak MR film overnight.

Autoradiographic visualization of amyloid plaques was observed in grey matter of postmortem AD brains. Plaque labeling with ¹²³I-1b-3 colocalize with plaque staining by Congo Red.

### Organ Distribution in Normal Mice

While under isoflurane anesthesia, 0.15 ml of a 0.9% saline solution containing ¹²³I-1b-3 (5-10 μCi) was injected directly into the tail vein of ICR mice (22-25 g, male and female) The mice (n=3 for each time point per sex) were sacrificed, the organs of interest were removed, weighed, and assayed with an automatic gamma counter. The percentage dose per organ was calculated by a comparison of the tissue counts to suitably diluted aliquots of the injected material. Total activities of blood were calculated under the assumption that they were 7% of the total body weight. The % dose/g of samples was calculated by comparing the sample counts with the count of the diluted initial dose. The results (Table 6) suggest ¹²³I-1b-3 readily penetrates and is rapidly cleared from the brain of normal mice.

TABLE 6
Biodistribution of ¹²³I-1b-3 after an i.v. injection in normal male mice
(average of 3 mice ± SD) % dose/organ and % dose/g of blood,
muscle, skin and bone were calculated under the assumption that these tissues
represent 7%, 40%, 15% and 14% of body mass respectively.

| % dose/organ | | | | |
|---|---|---|---|---|
| Organ | 2 min | 60 min | 120 min | 180 min |
| Blood | 7.96 ± 0.37 | 4.36 ± 0.62 | 3.78 ± 0.39 | 2.85 ± 0.99 |
| Heart | 1.08 ± 0.12 | 0.23 ± 0.04 | 0.16 ± 0.02 | 0.14 ± 0.04 |
| Muscle | 21.26 ± 3.15 | 7.20 ± 0.87 | 6.73 ± 1.64 | 5.51 ± 1.14 |
| Lung | 1.15 ± 0.16 | 0.40 ± 0.03 | 0.34 ± 0.04 | 0.43 ± 0.19 |
| Kidney | 4.44 ± 0.48 | 1.60 ± 0.07 | 0.89 ± 0.10 | 0.73 ± 0.11 |
| Spleen | 0.50 ± 0.13 | 0.28 ± 0.07 | 0.23 ± 0.06 | 0.20 ± 0.05 |
| Liver | 22.33 ± 4.22 | 19.37 ± 2.08 | 13.93 ± 0.86 | 11.74 ± 1.26 |
| Skin | 3.05 ± 0.27 | 5.51 ± 1.49 | 5.09 ± 1.50 | 3.73 ± 1.24 |
| Brain | 1.01 ± 0.16 | 0.26 ± 0.05 | 0.15 ± 0.01 | 0.12 ± 0.01 |
| Bone | 6.15 ± 0.34 | 4.17 ± 1.34 | 3.03 ± 1.41 | 2.22 ± 0.27 |
| Thyroid | 0.04 ± 0.01 | 0.77 ± 0.42 | 2.23 ± 1.66 | 1.72 ± 0.70 |
| Pancreas | 0.71 ± 0.13 | 0.32 ± 0.09 | 0.21 ± 0.03 | 0.19 ± 0.06 |
| Stomach | 1.03 ± 0.04 | 6.55 ± 2.11 | 9.84 ± 4.04 | 5.04 ± 2.04 |
| Intestine | 6.18 ± 0.78 | 31.30 ± 5.67 | 36.29 ± 9.61 | 44.10 ± 9.73 |
| Urogenital system | 0.40 ± 0.11 | 0.86 ± 0.12 | 0.98 ± 0.20 | 0.44 ± 0.10 |
| Testes | 0.14 ± 0.02 | 0.19 ± 0.02 | 0.14 ± 0.04 | 0.17 ± 0.03 |
| Tail | 15.52 ± 7.86 | 2.51 ± 0.76 | 2.23 ± 0.82 | 2.11 ± 0.32 |
| Fat | 0.28 ± 0.11 | 0.88 ± 0.12 | 0.75 ± 0.15 | 0.91 ± 0.20 |
| Carcass | 23.10 ± 6.51 | 14.89 ± 2.92 | 12.81 ± 3.00 | 9.37 ± 1.92 |
| % total recovered | 82.20 ± 6.38 | 83.26 ± 6.99 | 83.56 ± 6.29 | 79.15 ± 6.31 |

Table 6b:
Biodistribution of ¹²³I-1b-3 after an i.v. injection in normal male mice
(average of 3 mice ± SD) % dose/organ and % dose/g of blood,
muscle, skin and bone were calculated under the assumption that these tissues
represent 7%, 40%, 15% and 14% of body mass respectively.

| % dose/g | | | | |
|---|---|---|---|---|
| Organ | 2 min | 60 min | 120 mi | 180 min |
| Blood | 3.63 ± 0.29 | 2.04 ± 0.26 | 1.80 ± 0.24 | 1.37 ± 0.45 |
| Heart | 7.24 ± 1.41 | 1.55 ± 0.24 | 1.20 ± 0.19 | 1.00 ± 0.30 |
| Muscle | 1.70 ± 0.31 | 0.59 ± 0.08 | 0.56 ± 0.12 | 0.47 ± 0.10 |
| Lung | 6.38 ± 1.08 | 1.99 ± 0.20 | 1.81 ± 0.31 | 1.86 ± 0.77 |
| Kidney | 9.00 ± 1.79 | 3.12 ± 0.40 | 2.11 ± 0.28 | 1.65 ± 0.16 |
| Spleen | 4.86 ± 2.28 | 2.31 ± 0.52 | 2.34 ± 0.71 | 1.92 ± 0.33 |
| Liver | 11.83 ± 3.10 | 10.26 ± 1.88 | 8.18 ± 1.36 | 7.23 ± 0.94 |
| Skin | 0.65 ± 0.05 | 1.20 ± 0.28 | 1.12 ± 0.31 | 0.84 ± 0.28 |
| Brain | 2.05 ± 0.41 | 0.51 ± 0.09 | 0.31 ± 0.04 | 0.25 ± 0.03 |
| Bone | 1.40 ± 0.11 | 0.97 ± 0.26 | 0.72 ± 0.35 | 0.53 ± 0.05 |
| Thyroid | 4.63 ± 1.21 | 53.91 ± 27.07 | 119.16 ± 18.93 | 185.24 ± 49.80 |
| Pancreas | 3.52 ± 0.18 | 1.55 ± 0.39 | 1.20 ± 0.19 | 0.94 ± 0.24 |
| Stomach | 1.92 ± 0.23 | 9.48 ± 2.75 | 14.81 ± 8.53 | 9.92 ± 4.54 |
| Intestine | 2.07 ± 0.34 | 10.32 ± 1.43 | 12.37 ± 3.08 | 14.96 ± 3.85 |
| Urogenital system | 1.05 ± 0.17 | 3.03 ± 0.24 | 3.29 ± 0.92 | 1.79 ± 0.41 |
| Testes | 0.62 ± 0.09 | 0.86 ± 0.06 | 0.77 ± 0.22 | 0.70 ± 0.10 |
| Tail | 19.73 ± 10.26 | 3.59 ± 1.05 | 3.20 ± 1.08 | 3.03 ± 0.50 |
| Fat | 0.65 ± 0.27 | 2.21 ± 0.53 | 1.76 ± 0.16 | 2.18 ± 0.46 |
| Carcass | 1.38 ± 0.15 | 0.96 ± 0.03 | 0.83 ± 0.04 | 0.65 ± 0.15 |

### Partition Coefficient

Partition coefficients were measured by mixing ¹²³I-1b-3 with 3 g each of 1-octanol and buffer (0.1 M phosphate, pH 7.4) in a test tube. The test tube was vortexed for 3 min at room temperature, followed by centrifugation for 5 min. Two weighed samples (0.5 g each) from the 1-octanol and the buffer layer were counted in a well counter. The partition coefficient was determined by calculating the ratio of cpm/g of 1-octanol to that of the buffer. Samples from the 1-octanol layer were repartitioned until consistent partitions of coefficient values were obtained from the third partition. The measurement was done in triplicate and repeated three times. The partition coefficient for ¹²³I-1b-3 was determined to be 432 (logP=2.63).

### Example 3

A compound of the present invention is tested in an established in-vitro immunoblot assay for its ability to inhibit the formation of Aβ oligomers and fibrils (Yang F, Liim G P, Begum A N et al. Curcumin inhibits formation of amyloid β oligomers and fibrils, binds plaques, and reduces amyloid in-vivo. J. Biol. Chem. 280:5892-5901, 2005). Curcumin, a natural molecule serves as positive control. Compounds of this invention are able to inhibit the aggregation Aβ in a manner similar to Curcumin at concentrations of 1-100 μM.

It will be understood to those of ordinary skill in the art that the same can be performed within a wide and equivalent range of conditions, formulations, and other parameters without affecting the scope of the invention or any embodiment thereof. All patents, patent applications, and publications cited herein are fully incorporated by reference herein in their entirety.

What is claimed:

1. A compound of Formula I: 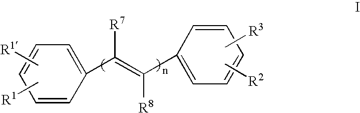
or a pharmaceutically acceptable salt thereof, wherein,
n is an integer from 1 to 6;
R¹ and R^{1†} are each independently:
-(CH₂)ₚNR^{a}R^{b}, wherein R^{a} and R^{b} are independently hydrogen, (C₁₋₄)alkyl, hydroxy(C₁₋₄)alkyl or halo(C₁₋₄)alkyl, and p is an integer from 0 to 5; hydroxy;
(C₁₋₁₄)alkoxy; hydroxy(C₁₋₄)alkyl; halogen; cyano; hydrogen; nitro; (C₁-C₄)alkyl; halo(C₁-C₄)alkyl; formyl; —NHCO(C₁₋₄ alkyl), or
—OCO(C₁₋₁₄ alkyl);
R³ is radiohalogen, radiohalo(C₁₋₄)alkyl, Si(C₁₋₄ alkyl)₃ or —Sn(C₁₋₄ alkyl)₃;
R² is: 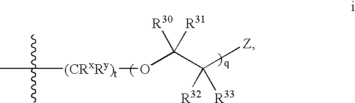
wherein q is an integer from 1 to 10, R^{x} and R^{y} are hydrogen, hydroxy or (C₁₋₄)alkyl; t is 0, 1, 2 or 3; Z is hydrogen, hydroxy, halogen, (C₁₋₄)alkoxy, (C₁₋₄)alkyl, and hydroxy(C₁₋₄)alkyl, and R³⁰, R³¹, R³² and R³³ are in each instance independently hydrogen, hydroxy, (C₁₋₄)alkoxy, (C₁₋₄)alkyl, or hydroxy(C₁₋₄)alkyl; 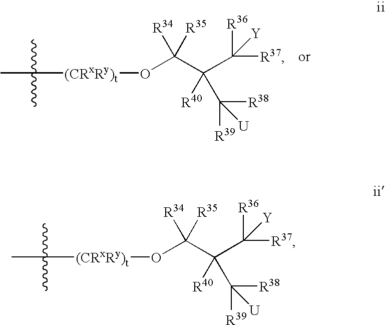
wherein R^{x} and R^{y} are hydrogen, hydroxy or (C₁₋₄)alkyl; t is 0, 1, 2 or 3; Y is hydrogen, hydroxy, halogen, (C₁₋₄)alkoxy, (C₁₋₄)alkyl, or hydroxy(C₁₋₄)alkyl; U is hydrogen, hydroxy, halogen, halogen substituted benzoyloxy, halogen substituted phenyl(C₁₋₄)alkyl, halogen substituted aryloxy, or halogen substituted (C₆₋₁₀)aryl; and R³⁴, R³⁵, R³⁶, R³⁷, R³⁸, R³⁹ and R⁴⁰ are in each instance independently hydrogen, halogen, hydroxy, (C₁₋₄)alkoxy, (C₁₋₄)alkyl, or hydroxy(C₁₋₄)alkyl;
iii. NR^{\}R^{\\}, wherein at least one of R^{†} and R^{††} is (CH₂)_{d}X, where X is halogen, preferably F or ¹⁸F, and d is an integer from 1 to 4; the other of R^{†} and R^{††} is hydrogen, (C₁₋₄)alkyl, halo(C₁₋₄)alkyl, or hydroxy(C₁₋₄)alkyl;
iv. NR^{\}R^{\\}-(C₁₋₄)alkyl, wherein at least one of R^{†} and R^{††} is (CH₂)_{d}X, where X is halogen, preferably F or 18F, and d is an integer from 1 to 4; the other of R^{†} and R^{††} is hydrogen, (C₁₋₄)alkyl, halo(C₁₋₄)alkyl, or hydroxy(C₁₋₄)alkyl;
v. halo(C₁₋₄)alkyl; or
vi. an ether (R—O—R) having the following structure: [halo(C₁₋₄)alkyl-O—(C₁₋₄)alkyl]-;
and
R⁷ and R⁸ are in each instance independently hydrogen, hydroxy, amino, methylamino, dimethylamino, (C₁₋₄)alkoxy, (C₁₋₄)alkyl, or hydroxy(C₁₋₄)alkyl.

2. The compound of claim 1 wherein, R³ is ¹²⁵I, ¹²³I, ¹³¹I, ¹²⁴I, ¹⁸F, ¹⁸F(C₁₋₄ alkyl), ⁷⁶Br, ⁷⁷Br, Si(C₁₋₄ alkyl)₃, or —Sn(C₁₋₄ alkyl)₃.

3. The compound of claim 1 wherein, R¹ is hydroxy, —OCO(C₁₋₄alkyl) or —(CH₂)ₚNR^{\}R^{\\}, wherein R^{†} and R^{††} are independently hydrogen, (C₁₋₄)alkyl, hydroxy(C₁₋₄)alkyl or halo(C₁₋₄)alkyl, and p is an integer from 0 to 5.

4. The compound of claim 1 wherein, R¹ is hydroxy or —(CH₂)ₚNR^{\}R^{\\}, wherein R^{†} and R^{††} are independently hydrogen or (C₁₋₄)alkyl, and p is 0.

5. The compound of claim 1 wherein, R^{1\} is hydrogen.

6. The compound of claim 1 wherein, t is 0 and R² is 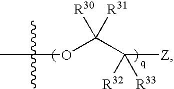
wherein q is an integer from 1 to 4; Z is halogen or hydroxy, and R³⁰, R³¹, R³² and R³³ are in each instance independently hydrogen, hydroxy, (C₁₋₄)alkoxy, (C₁₋₄)alkyl, or hydroxy(C₁₋₄)alkyl.

7. The compound of claim 6 wherein, Z is fluoro and R³⁰, R³¹, R³² and R³³ are in each instance hydrogen.

8. The compound of claim 1 wherein, t is 0, and R² is

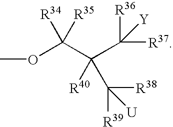

9. The compound of claim 8 wherein, U is hydroxy.

10. The compound of claim 8 wherein, Y is halogen or hydrogen.

11. The compound of claim 8 wherein, R³⁴, R³⁵, R³⁶, R³⁷, R³⁸, R³⁹ and R⁴⁰ are in each instance hydrogen.

12. The compound of claim 1 having the following structure:

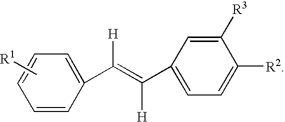

13. The compound of claim 12, wherein
R¹ is hydroxy, —OCO(C₁₋₄)alkyl, —NHCO(C₁₋₄)alkyl, or —(CH₂)ₚNR^{\}R^{\\}, wherein R^{†} and R^{††} are independently hydrogen, (C₁₋₄)alkyl, hydroxy(C₁₋₄)alkyl or halo(C₁₋₄)alkyl, and p is an integer from 0 to 5; and R² is ii.

14. The compound of claim 13, wherein R³ is a radiohalogen or —Sn(C₁₋₄ alkyl)₃.

15. The compound of claim 14, wherein R³ is ¹²³I.

16. The compound of claim 1, having one of the following structures: 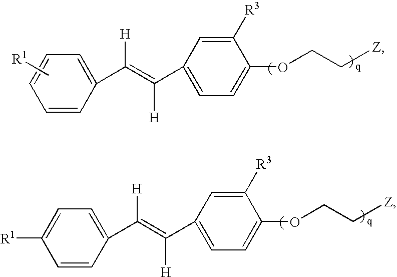
wherein q is an integer from 2 to 4 and Z is hydroxy or halogen; 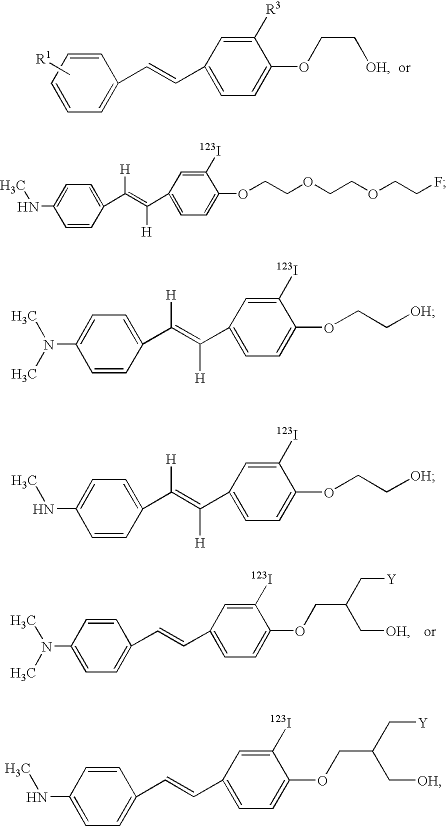
wherein Y is hydrogen or F.

17. The compound of claim 1 that is:

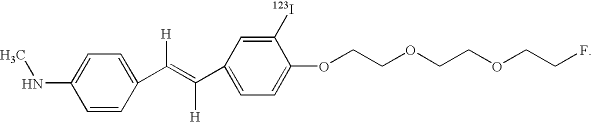

18. The compound of claim 1 that is:

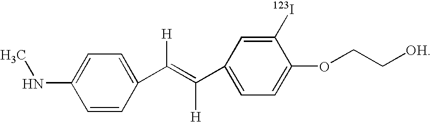

19. A pharmaceutical composition comprising a compound of claim 1.

20. A diagnostic composition for imaging amyloid deposits, comprising a radiolabeled compound of claim 1.

21. A method of imaging amyloid deposits, comprising:
a. introducing into a mammal a detectable quantity of the diagnostic composition of claim 20;
b. allowing sufficient time for the labeled compound to be associated with amyloid deposits; and
c. detecting the labeled compound associated with one or more amyloid deposits.

22. A method of inhibiting amyloid plaque aggregation in a mammal, comprising administering the composition of claim 19 to the mammal in an amount effective to inhibit amyloid plaque aggregation.
